(19) United States
(12) Patent Application Publication
(10) Pub. No.: US 2008/0160638 A1
(43) Pub. Date: Jul. 3, 2008

(54) Functionalized Microcantilever Sensor and Associated Method For Detection of Targeted Analytes

(76) Inventors: David Lederman, Morgantown, WV (US); Daniel Flynn, Morgantown, WV (US); Peter Mico Gannett, Morgantown, WV (US); Odille Cue Noriega Myers, Morgantown, WV (US)

Correspondence Address: JOHN E. GROSSELIN, III;SPILMAN THOMAS & BATTLE PLLC, C/O INTELLEVATE, P.O. BOX 52050, MINNIAPOLIS, MN, 55402, US

(21) Appl. No.: 11/962,229

(22) Filed: Dec. 21, 2007

Related U.S. Application Data
(60) Provisional application No. 60/877,255, filed on Dec. 27, 2006.

(51) Int. Cl. G01N 33/553; B01J 19/00; B05D 1/36; G01N 33/00; B05D 7/24; G03F 7/00

(52) U.S. Cl. 436/525; 422/68.1; 427/402; 427/414; 427/419.1; 427/404; 430/296; 436/94; 436/86

(57) ABSTRACT

A microcantilever sensor for targeted analyte detection can generally comprise a microcantilever having a base and a beam, a metallic coating disposed substantially only on a first surface of a distal-most end of the beam, and a receptor compound immobilized to the metallic coating wherein the receptor compound can have substantially exclusive binding interaction with the analyte. The receptor compound can be a thiol-terminated bifunctional compound having a receptor site with specific binding affinity for the analyte, for example, an isolated Fab′ fragment. The metallic coating can be a noble metal and/or a semi-noble metal, such as a bilayer of chromium and gold. The metallic coating can be applied to the microcantilever surface by electron-beam lithography.

### CROSS REFERENCE TO RELATED APPLICATION

This application claims priority to U.S. Provisional Patent Application Ser. No. 60/877,255, filed Dec. 27, 2006, which is hereby incorporated by reference in its entirety.

### BACKGROUND

The functionalized microcantilever sensor generally relates to microsensors, and in particular, functionalized microcantilever sensors and associated methods for detection of targeted analytes.

Microcantilever resonators have been shown to be extremely sensitive biosensors. In this technique, similar to measurements performed earlier on quartz crystal monitors where small changes in mass can be detected, both the change in resonance frequency and deflection of the cantilever, due to mass change or strain induced on the cantilever as a result of analyte-specific binding, can be used as detection schemes. For example, the microcantilever deflection in solution has been successfully demonstrated to detect antibodies and the prostate cancer marker protein PSA. State-of-the-art enzyme linked immunosorbent assays (ELISA) techniques, although not necessarily those available in standard laboratories, indicate detection levels in the range of 10-100 pg/ml. Even lower detection levels of approximately 10 fg/ml have been reported using ELISA with multiphoton detection. However, microcantilever detection technology can be superior to ELISA in that it does not require labels for analyte detection or multiple steps with separate reagents. In addition, microcantilever technology readily lends itself to the formation of microarrays using well-known microfabrication techniques, thereby offering the promising prospect of becoming a high-throughput protein analysis platform.

Vascular endothelial growth factor (VEGF) is a protein associated with tumor growth and an important protein marker for lung, cervical, and other cancers. In particular, the current standard treatment of lung cancer consists of chemotherapy and radiation therapy. Although both of these treatments improve patient survival, both are nonspecific and toxic. Because the highest survival rates are associated with surgical removal of the tumor at an early stage of lung cancer, early detection is crucial to increase survival rates. Unfortunately, at present, most lung cancer patients are diagnosed at advanced stages of cancer. New molecular-targeted techniques that can be inexpensively and routinely applied are currently being developed for early detection of lung cancer tumors. Current obstacles include identifying at-risk patient populations, identifying biomarkers that are predictive of early disease, and detection of very low levels of these biomarkers. One potential biomarker for lung cancer is VEGF.

VEGF is one of the most important angiogenic factors that are expressed in a large number of human cancers. VEGF is a cytokine secreted by tumors that promotes angiogenesis, or the formation of new blood vessels from pre-existing ones. VEGF is also involved in many other physiological and pathological conditions. Tissues, including tumors, interact with the vasculature in order to receive oxygen, nutrients, and growth factors, and dispose of waste products. Since tumor size is limited to less than 2 mm without an adequate blood supply that can support tumor growth, angiogenesis is required for tumor growth and progression. In fact, tumor progression to malignancy is characterized by changing from an avascular state to an angiogenic phase that is marked by the recruitment of new blood vessels. High expression of VEGF is found in nearly all types of cancer. Also, an increased level of tumor-associated VEGF seems to correlate with disease recurrence and decreased survival rates in cancer patients. Elevated VEGF levels can be detected in tumor biopsies and body fluids. In lung cancer patients, elevated levels of VEGF are present in serum and, in some studies, high levels of expression correlate with decreased survival rates. Elevated VEGF levels, however, can also be detected in sputum of patients with respiratory inflammatory diseases, such as asthma and emphysema.

Nevertheless, when VEGF is combined with other protein markers, including circulating VEGF (VEGF-C) and matrix metalloproteinase 9 (MMP-9), the sensitivity, or the ability to detect the cancer, and the specificity, or the ability to differentiate between cancer and a benign tumor, are greater than 90% and 70%, respectively. Thus, methods to detect and quantify VEGF and related proteins may significantly improve the likelihood of early detection of lung cancer, and therefore, successful treatment. Current laboratory detection strategies use ELISA where nanogram levels can be detected in serum. It is predicted that if much lower but significant levels of VEGF could be detected, VEGF could be used as an indicator for early disease, especially if correlated with the presence of other proteins, such as MMP-9. If early signs of disease can be detected, then patients at risk for diseases, such as lung cancer, could be triaged to imaging techniques, such as spiral CT, where small tumors could be visualized. Tumors detected at an early phase of development respond more favorably to standard care.

### SUMMARY

A microcantilever sensor for targeted analyte detection can generally comprise a microcantilever having a base and a beam, a metallic coating disposed substantially only on a first surface of a distal-most end of the beam, and a receptor compound immobilized to the metallic coating wherein the receptor compound can have substantially exclusive binding interaction with the analyte. In certain embodiments, the receptor compound can be a thiol-terminated bifunctional compound having an active receptor site with specific binding affinity for the analyte. The receptor compound can be, for example, a biochemical receptor compound, such as an antibody, an isolated Fab′ fragment, a DNA fragment, a RNA fragment, an aptamer, a protein, a carbohydrate, and DTSP. The metallic coating can be at least one of a noble metal and a semi-noble metal, such as, for example, a bilayer of chromium and gold. The said metallic coating can be disposed in a pattern having a total area from about 100 μm² to about 144 μm². The metallic coating can be disposed in a square or rectangular pattern having an edge length in the range of about 10 μm to about 12 μm. The metallic coating can be applied to the microcantilever by electron-beam lithography. In a further embodiment, the microcantilever sensor can further comprise a plurality of microcantilevers sensors disposed in an array.

In another embodiment, a method for functionalizing a microcantilever sensor for targeted analyte detection can generally comprise disposing a metallic coating substantially only on a first surface of a distal-most end of the microcantilever and immobilizing a receptor compound to the metallic coating wherein the receptor compound can have substantially exclusive binding interaction with the analyte. In certain embodiments, the receptor compound can be a thiol-terminated bifunctional compound having an active receptor site with specific binding affinity for the analyte. The receptor compound can be, for example, a biochemical receptor compound, such as an antibody, an isolated Fab′ fragment, a DNA fragment, a RNA fragment, an aptamer, a protein, a carbohydrate, and DTSP. The metallic coating can be at least one of a noble metal and a semi-noble metal, such as, for example, a bilayer of chromium and gold. The said metallic coating can be disposed in a pattern having a total area from about 100 μm² to about 144 μm². The metallic coating can be disposed in a square or rectangular pattern having an edge length in the range of about 10 μm to about 12 μm. The metallic coating can be applied to the microcantilever by electron-beam lithography.

In a further embodiment, a method for using a microcantilever sensor for detecting a targeted analyte can generally comprise treating a first surface of a distal-most end of the microcantilever with a metallic compound, immobilizing a receptor compound to the metallic compound wherein the receptor compound can have substantially exclusive binding interaction with the targeted analyte, exposing the first surface to a sample solution containing the targeted analyte, drying the first surface, and resonating the microcantilever in air or vacuum to detect the targeted analyte. In certain embodiments, the receptor compound can be a thiol-terminated bifunctional compound having an active receptor site with specific binding affinity for the analyte. The receptor compound can be, for example, a biochemical receptor compound, such as an antibody, an isolated Fab′ fragment, a DNA fragment, a RNA fragment, an aptamer, a protein, a carbohydrate, and DTSP. The metallic coating can be at least one of a noble metal and a semi-noble metal, such as, for example, a bilayer of chromium and gold. The said metallic coating can be disposed in a pattern having a total area from about 100 μm² to about 144 μm². The metallic coating can be disposed in a square or rectangular pattern having an edge length in the range of about 10 μm to about 12 μm. The metallic coating can be applied to the microcantilever by electron-beam lithography.

### BRIEF DESCRIPTION OF THE DRAWING FIGURES

A more complete understanding of the functionalized microcantilever sensor and associated methods can be obtained by considering the following detailed description in conjunction with the accompanying drawing figures, in which:

FIG. 1(a) is a schematic of an embodiment of the cantilever after each step of the functionalization and chemical binding process.

FIG. 1(b) is a schematic of an embodiment of the cantilever after each step of the functionalization and chemical binding process.

FIG. 2(a) shows resonance profiles indicating the resonance frequency shift of another embodiment of a bare cantilever after coating with APTES, glutaraldehyde, and PA.

FIG. 2(b) shows the resonance frequency shift after coating the cantilever of FIG. 2(a) with anti-VEGF.

FIG. 2(c) shows the resonance frequency measurement histogram of the bare cantilever.

FIG. 2(d) shows the resonance frequency measurement histogram after coating with PA.

FIG. 2(e) shows the resonance frequency measurement histogram after coating with anti-VEGF.

FIG. 3(a) shows resonance frequency profiles of the cantilever of FIG. 2(b) after coating with BSA and subsequent exposure to ferritin and VEGF.

FIG. 3(b) shows the resonance frequency measurement histogram after exposure to BSA.

FIG. 3(c) shows the resonance frequency measurement histogram after exposure to ferritin.

FIG. 3(d) shows the resonance frequency measurement histogram after exposure to VEGF.

FIG. 4(a) shows resonance profiles of an embodiment of a cantilever after each step of the functionalization and chemical binding process as indicated in the graphs.

FIG. 4(b) shows resonance profiles of an embodiment of a cantilever after each step of the functionalization and chemical binding process as indicated in the graphs.

FIG. 5 shows the measured mass of VEGF bound to the cantilever as a function of nominal solution concentration.

FIG. 6 shows ELISA measurements of fluorescence at a wavelength of 450 nm for incubation times of 3 min, 6 min, and 9 min.

FIG. 7(a) is a side view of an embodiment of a microcantilever sensor.

FIG. 7(b) is a top view of the microcantilever sensor of FIG. 7(a).

FIG. 8(a) is a side view of an embodiment of the cantilever assembly after spin coating with PMMA.

FIG. 8(b) is a side view of the cantilever assembly of FIG. 8(a) after developing and exposure.

FIG. 8(c) is a side view of the cantilever assembly of FIG. 8(b) after deposition of the metallic coating.

FIG. 8(d) is a side view of the cantilever assembly of FIG. 8(c) after the lift off process.

FIG. 9 is an optical microscopy image of an embodiment of a microcantilever with a gold pad deposited and defined at the distal-most end of the cantilever.

FIG. 10 is an atomic force microscopy image analysis of a gold pad deposited at the distal-most end of a silicon surface.

FIG. 11 is an atomic force microscopy image analysis of the gold pad of FIG. 10 after treating with DTSP.

FIG. 12 is a summary representation of modifying Fab′ antibody (IgG) segments according to certain embodiments of the invention.

FIG. 13 is a summary representation of immobilizing Fab′ fragments onto a gold surface according to certain embodiments of the invention.

### DESCRIPTION OF CERTAIN EMBODIMENTS

Although the functionalized microcantilever sensor and associated method are described in connection with the specific detection of vascular endothelial growth factor (VEGF) and (matrix metalloproteinase 9) MMP-9, it is to be understood that the microcantilever sensor and methods described herein can also be designed and used to detect other targeted analytes and compounds. The methodology relies on defining a receptor site at the distal-most end of a cantilever beam by selecting a bifunctional receptor compound that provides specific binding to the targeted analyte and selectively adhering this receptor compound only to a first surface of the distal-most end of the cantilever beam and not to the entire cantilever.

Since VEGF is an important marker for various types of cancers, its detection with sufficient specificity and sensitivity under clinically relevant conditions would be a significant advancement in the detection and ultimate treatment of the disease. Hereinafter, it is demonstrated that a microcantilever detection technique can be useful for the sensitive and specific detection of protein markers for cancer, for example, VEGF. A monoclonal anti-VEGF antibody can be immobilized to a silicon cantilever surface and the shift of the cantilever resonance frequency due to VEGF binding with this antibody can be measured to detect VEGF in solution. The strong antigen-antibody binding allows measurements in air, which increases the sensitivity of the device by reducing damping present in solution, thus increasing the resonance frequency and reducing the width of the resonance.

Furthermore, the potential problem of interference from non-VEGF proteins was reduced by treating the cantilevers with bovine serum albumin (BSA). The selectivity of the microcantilever sensor was validated by exposing the microcantilever sensor to proteins other than VEGF, e.g., ferritin and MMP-9, where no significant frequency shift was observed to within a sensitivity of approximately 0.2 pg. The resonance frequency of an anti-MMP-9-coated cantilever also changed when exposed to MMP-9. These results indicated that nonspecific binding can be minimized. A calibration of the cantilevers yielded a minimum detectible concentration of approximately 6.3 pg/ml. In comparison, ELISA measurements yielded a minimum sensitivity of approximately 7 pg/ml. Test results indicated that selectively functionalizing only a portion of the cantilever so that the targeted analyte or compound only bind to that area enables the measurement of small concentrations of analytes or compounds in small volumes of solution. This ability to rapidly detect very low levels of biomarkers may be useful for early detection of cancer.

Unless otherwise noted, all chemicals and solvents were purchased from SIGMA (St. Louis, Mo.). Water was obtained from a BARNSTEAD NANOPURE water purification system (Dubuque, Iowa).

Commercial silicon, tipless, rectangular AFM cantilevers 90 μm long, 35 μm wide, and 2 μm thick obtained from MIKROMASCH USA (Wilsonville, Oreg.) were used in the experiment. The silicon cantilever was mounted on a holder and fixed into a commercially available atomic force microscope system, MULTIMODE model by VEECO (Santa Barbara, Calif.). A low-power diode laser (3 mW) was focused onto the cantilever. The reflected laser beam was directed onto a position-sensitive detector that can detect the deflection amplitude of the cantilever. A piezoelectric ceramic plate mounted on the cantilever holder was used to generate the oscillation of the cantilever. The resonance frequency was determined from the maximum amplitude of the cantilever. The resonance frequency is not sensitive to bending due to strain; rather, it is only sensitive to any change in mass.

The resonance frequency (f₀) and spring constant (K) specified by the vendor were approximately 300 kHz and 15 N/m, respectively. In order to determine these values exactly for each cantilever, f₀ was measured for bare cantilevers in air. In air or under vacuum conditions, the resonance frequency of the cantilever is given by equation 1:

$${f}_{0} = \frac{1}{2 π} \sqrt{\frac{K}{{m}^{*}}} \qquad ( 1 )$$

where m* is the effective mass of the cantilever. For a rectangular cantilever, m*=0.24 m, where m is the mass of the cantilever, which can be calculated from the density of silicon and the cantilever's dimensions. The spring constant was calculated from Equation 1 by measuring the resonance frequency of the bare cantilever and solving for K.

### Overview

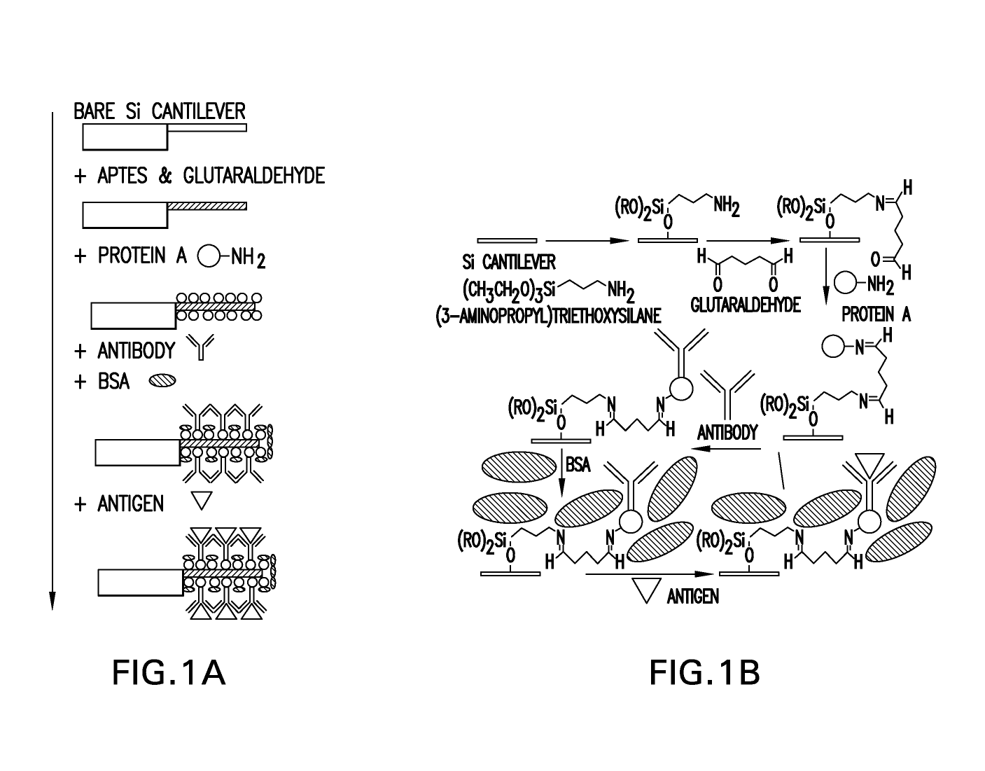

Referring now to the drawing figures, wherein like reference numerals are used to refer to like elements throughout, a schematic of an embodiment of the functionalization and chemical binding process is illustrated in FIGS. 1(a) and 1(b), in which R may be alkyl, chloro, or Si groups depending on the formation of the self-assembled monolayer. During the cantilever chemical functionalization process, a custom fabricated TEFLON (DUPONT, Wilmington, Del.) cantilever holder was created to secure the cantilever and prevent it from breaking. The fungal immobilization on cantilevers is known. See, e.g., Nugaeva, N., Gfeller, K. Y., Backmann, N., Lang, H. P., Duggelin, M., Hegner, M. “Micromechanical cantilever array sensors for selective fungal immobilization and fast growth detection”, Biosensors and Bioelectronics 21:849-856 (2005). Briefly, the cantilever was silanized with 3-aminopropyltriethoxysilane (APTES, 1% wt. toluene), an amino-terminated silane that tends to self-assemble in a monolayer. The cantilever then was coated with glutaraldehyde (2.5% wt. solution in water) for 1 hour at 24° C. in phosphate buffered saline (PBS, 0.15 M, pH 7.4). Glutaraldehyde is frequently used in biochemistry applications as an amine-reactive homobifunctional crosslinker. However, any α,ω-homobifunctional crosslinker with the structure X—R—X, wherein X can be any functionality that is reactive toward amines, carboxylates, or thiols, and R can by alkyl or aryl, could be substituted for glutaraldehyde. Then the cantilever was rinsed in water and dried in air in order to avoid the stiction problem that normally occurs for micromachined surface structures after being pulled from a liquid medium. Drying the cantilevers also helped remove excess water adsorption that would alter the mass measurement in a non-reproducible manner.

In one embodiment, the APTES and glutaraldehyde-coated cantilever was rinsed and then reacted with Protein A (PA, Staphylococcus aureus, Cowan Strain, 100 μg/mL) for 12 hours at 4° C. in PBS to chemically link PA to the remaining aldehyde group of glutaraldehyde. The stability of the linkage can be improved by chemical reducing the imino-crosslink with sodium borohydride to an amino-crosslink. See, e.g., Nam, S.-H. and Walsh, M. K. “Covalent immobilization of bovine phospholipase A2”, J. Food. Biochemistry, 29:1-12 (2005). PA binds with high affinity to Immunoglobulin G (IgG) of numerous mammalian species via the Fc portion of the antibody. PA is widely used in both research and bioprocesses, such as the development of biosensors and antibody purification protocols. The surface-bound IgG must be immobilized with its Fab′-localized antigen-binding sites accessible to antigens in order for the IgG to be effective in immunoassay techniques. PA was used to bind IgG isotype anti-VEGF with exposed Fab′ segments. The PA-coated cantilever was treated with a solution of monoclonal anti-VEGF with an IgG1 isotype (available from ABCAM, Cambridge, Mass., 1.30 mg/mL) in potassium phosphate buffer (0.15 M pH 7.4) for 2 hours at 24° C. This version of anti-VEGF has been shown to be excellent for immunohistochemical localization of VEGF in paraffin sections, indicating the strong affinity between anti-VEGF and its antigen. In order to confirm the binding affinity between the PA on the cantilever surface and the anti-VEGF proteins from the solution, the resonance frequency shift of the same cantilever used for PA binding was measured in air after the cantilever anti-VEGF treatment.

In order to avoid VEGF binding to possible uncoated regions of the cantilever, thus resulting in nonspecific adsorption, the antibody-coated cantilevers were exposed to a blocking compound, e.g., bovine serum albumin (BSA, 5 μg/mL), by soaking for 4 hours at 24° C. in PBS. Then the cantilever was exposed to VEGF (available from EMD BIOSCIENCES, San Diego, Calif., 2.0 μg/ml in PBS), ferritin (equine spleen, 25 mg/ml in saline), or MMP-9 (available from EMD BIOSCIENCES, San Diego, Calif., 2.0 μg/ml in PBS) for 4 hours via immersion and dried. The cantilever was then washed with de-ionized water, dried, and the resonance frequency measured. Similar experiments were carried out to test binding specificity by binding polyclonal anti-MMP-9 (available from ABCAM, Cambridge, Mass., 50 μg/ml) instead of anti-VEGF.

The resonance frequency was measured after each binding step (PA, antibody, BSA, antigen). The mass of the bound material was calculated from the change in resonance frequency after each step. Using Equation 1, if the resonance frequency changes from f₁ to f₂, the change in mass is given by equation 2:

$$Δ m = \frac{K}{4 {π}^{2}} ( \frac{1}{{f}_{2}^{2}} - \frac{1}{{f}_{1}^{2}} ) \qquad ( 2 )$$

The value of K is determined from the bare cantilever measurement, thus assuming that it does not change upon binding. In principle this might not be completely true because the strain induced at the surface due to the binding of APTES is known to change K, e.g., on silicon nitride cantilevers coated on one side with gelatin exposed to humidity and coated with gold and exposed to Hg. In this case however, this change is probably negligible because (1) APTES coats both sides of the cantilever; (2) the initial value of K is roughly 200 times larger than in the case of the silicon nitride cantilevers, making the fractional change in frequency very small; and (3) increases in strain usually result in an increase of the resonance frequency, whereas here, only decreases of the resonance frequency were observed. The resonance frequency value for each measurement was acquired by fitting a Lorentzian profile to a single resonance peak and the final value was the average of all measurements. The uncertainty in the measurement was determined from the standard deviation of multiple measurements.

In order to calibrate the microcantilevers, measurements were carried out using the procedures described above on nominal VEGF-PBS solutions ranging between 500 pg/ml and 1500 pg/ml. For this part of the experiment, care must be taken to only immerse the cantilever beam and not the cantilever beam and silicon support chip base assembly in the VEGF solution. The sensitivity can be increased by factor of 1000 because the supporting chip's surface area is approximately 1000 times larger than the cantilevers' beam surface areas. Here, a long focal length microscope was used to only immerse the cantilever beam in the VEGF solution in order to quantify more accurately. The frequency after each binding step was measured.

For these experiments, the frequencies of three cantilevers attached to the same chip were used. In one embodiment, the supporting chip can be approximately 3 mm long and 500 μm thick and the cantilevers had lengths of 90 μm, 100 μm, and 110 μm, with the same width and thickness of 35 μm and 2 μm, respectively. Because of the difference in surface areas, the effective nominal concentration for each cantilever was the solution concentration normalized to the different surface areas of the cantilevers. Several of the cantilevers broke during this process due to the tendency of silicon to cleave resulting in an increase in frequency, i.e., a decrease in mass. Other cantilevers were damaged by cracking, and as a result, they showed multiple resonance peaks. The presence of cracks was verified using optical and scanning electron microscopy. Only cantilevers that were not obviously broken and measurements on cantilevers that resulted in a decrease in frequency after anti-VEGF and VEGF binding and did not show multiple resonance peaks were used for calibration. Only 4 of the 18 cantilevers were used for the final calibration analysis. This can be an intrinsic weakness of measurements performed in air because a relatively low yield of microcantilever sensors is traded for an increase in sensitivity.

A comparison of the microcantilever sensitivity with that of the ELISA technique was carried out. The recombinant VEGF-A concentration was detected using a sandwich ELISA. A sandwich ELISA requires two antibodies: a “capture” antibody that binds to the bottom of the ELISA plate and also binds VEGF, and a “detector” antibody that binds to VEGF at a different epitope than the capture antibody. The detector antibody in this case was conjugated to biotin. The biotin then was bound to a streptavidin-horse radish peroxidase (HRP) conjugate. The VEGF concentration was determined by measuring the amount of streptavidin-HRP conjugate bound using the calorimetric substrate tetramethylbenzidine (TMB). TMB develops a blue reaction product when reacted with peroxidase. A serial dilution of VEGF was performed that ranged from 10,000 pg/ml to 156 pg/ml.

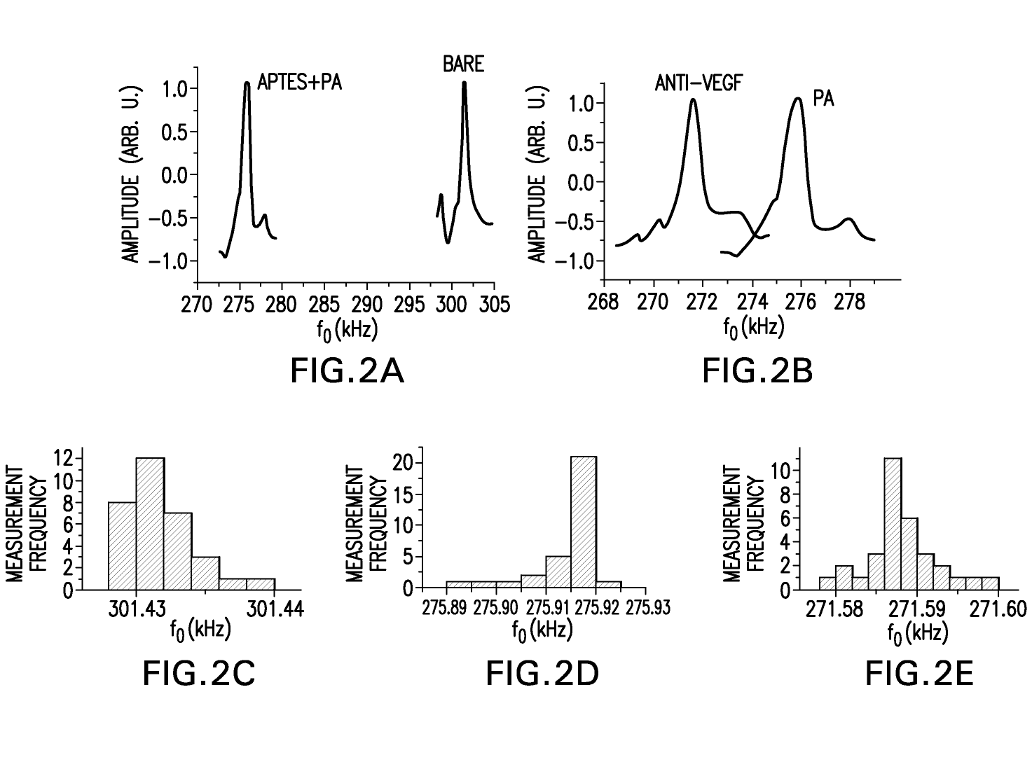

The shift of the resonance frequency after a cantilever was coated with the Protein A (PA) and dried is shown in FIG. 2(a). Measurements of each curve were repeated 30-times. The average resonance frequencies corresponding to the bare cantilever and PA-coated cantilever were 301.438 kHz±0.002 kHz and 275.914 kHz±0.005 kHz, respectively. The measurement distributions are shown in FIGS. 2(c)-(e). The spring constant of the cantilever was measured to be K=12.6 N/m. The added mass due to the APTES, glutaraldehyde, and PA coatings required to bind the antibody was calculated to be approximately 2.840 ng. Subsequently, when the PA-coated cantilever was ultrasonicated for 2 minutes in de-ionized water, no shift of the resonance frequency was observed. It was therefore assumed that PA molecules attach on the aldehyde-terminated siloxane surface predominantly by covalent bonding.

Referring to FIG. 2(b), there is a shift in the resonance frequency after the immersion of a PA coated cantilever in anti-VEGF solution. The average resonance frequency before anti-VEGF treatment, see FIG. 2(a), was 275.914 kHz, but shifted to 271.589 kHz±0.004 kHz after coating with anti-VEGF. The added mass was calculated to be 562 pg. It was assumed that anti-VEGF molecules bind to the PA layer. To confirm this assumption, the PA and anti-VEGF coated cantilever was ultrasonicated in de-ionized water for 2 minutes before and after the frequency measurement. Referring to FIGS. 2(c)-(e), the resonance frequency measurement historgram for the bare cantilever, the cantilever after coating with PA, and the cantilever after coating with anti-VEGF shows virtually no shift of the resonance frequency was observed after sonication.

The coverage of the anti-VEGF was determined from the surface area of the cantilever and the dimensions of the protein. The molecular weight of anti-VEGF is approximately 150 kDa and has an effective cross-sectional area of approximately 50 nm² (assuming anti-VEGF to be a sphere of approximately 8 nm in diameter), so that the maximum mass on the entire surface of the cantilever, including the sides, should be approximately 33 pg. Hence, the surface coverage ratio is approximately 17 times the expected value. The over-coverage could be due to roughness of the APTES binding layer, agglomeration of the antibody on the surface, or water molecules left in the proteins. Agglomeration on the cantilever surface was indeed observed with an optical microscope (data not shown).

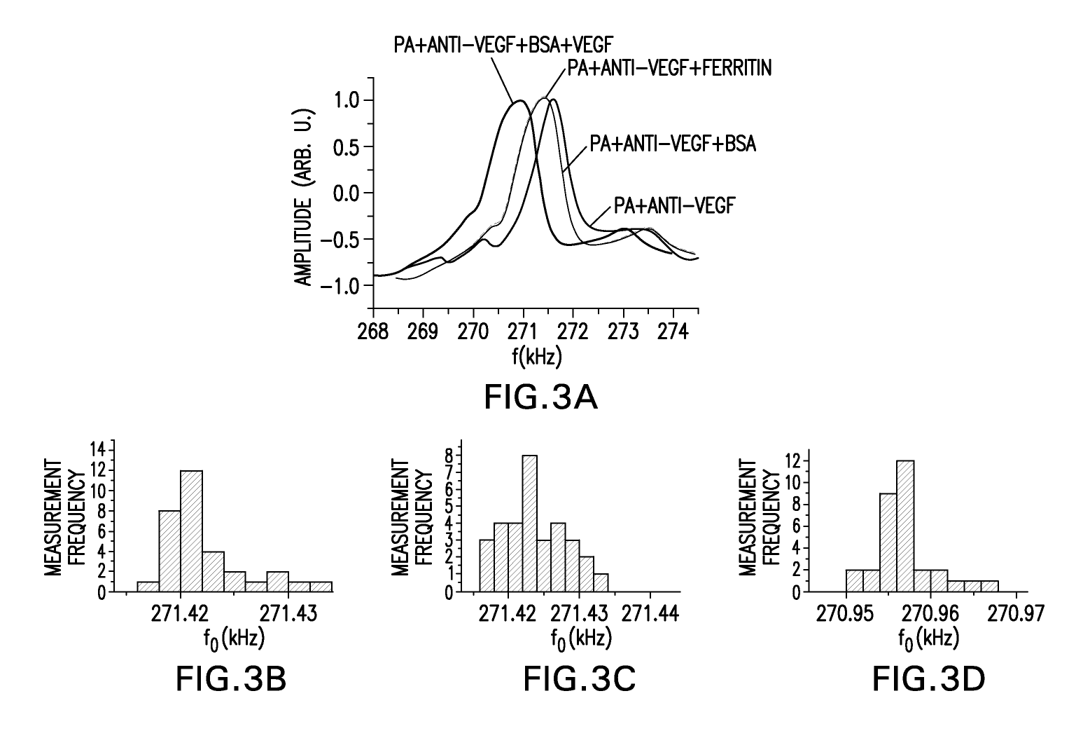

Referring to FIG. 3(a), the measured frequency response of the cantilever after BSA, ferritin, and VEGF exposures to the cantilever functionalized with anti-VEGF. As shown in FIG. 3(b), an average resonance frequency at 217.422 kHz after treatment of BSA corresponds to a mass change of 22 pg. The molecular weight of BSA is approximately 66 kDa with an effective area of approximately 56 nm² (assuming BSA to be an ellipsoid with dimensions of 14 nm by 4 nm). A calculation similar to that of the anti-VEGF yields 169% coverage, which, although large, is approximately ten times smaller than the anti-VEGF coverage. This indicates that the BSA covered approximately 10% of the area covered by the anti-VEGF. This number represents and upper limit of coverage since water molecules could be associated with BSA.

FIGS. 3(c) and 3(d) show the results after treating the anti-VEGF coated cantilever with VEGF and ferritin. The average resonance frequency after the ferritin treatment is 271.423 kHz, which is identical to the anti-VEGF resonance frequency to within the measurements' standard deviation of 0.004 kHz. This indicates that the average resonance frequency is effectively unchanged, and hence, there is no measurable anti-VEGF and ferritin specific binding. Then, after immersing the same cantilever into the VEGF solution, a significant frequency shift to 270.957 kHz was observed, as shown in FIG. 3(c), which corresponds to a mass increase of 62 pg. The molecular weight of VEGF is approximately 46 kDa, according to the manufacturer. Comparing this measurement with the anti-VEGF mass on the cantilever (562 pg), and given that the anti-VEGF protein mass is approximately three times larger than the VEGF mass, the binding corresponds to approximately 33%, that is, antigens were bound to approximately 33% of the antibodies.

The anti-VEGF and VEGF experiments were carried out on a second cantilever and the results are summarized in Table I (cantilevers 1 and 2). Table I is a summary of results for the cantilevers used in this study. In the table, f₀ is the resonance frequency measured in air of the bare cantilever, and K is the spring constant determined from f₀. MPA, MAB, MBSA, MAG, are the increases in mass detected due to the APTES, glutaraldehyde, and PA, antibody, BSA, and antigen adsorptions, respectively, calculated from Equation 2. The uncertainty in f₀ is approximately 0.004 kHz resulting in an uncertainty in the change in mass of approximately 0.2 pg. Although the mass change due to the APTES, glutaraldehyde, and PA coating was different, the amount of bound antibody and antigen is of the same order of magnitude for both cases. In the second case, the anti-VEGF and VEGF coverage was approximately 18%.

TABLE I
A summary of results for certain embodiments of the cantilevers used in this study.

| | | | | | M_{PA} | M_{AB} | M_{BSA} | M_{AG} |
|---|---|---|---|---|---|---|---|---|
| Cantilever | Antibody | Antigen | f₀ (kHz) | K (N/m) | (pg) | (pg) | (pg) | (pg) |
| 1 | Anti-VEGF | VEGF | 301.438 | 12.6 | 2,841 | 562 | 22.3 | 62.2 |
| 2 | Anti-VEGF | VEGF | 300.182 | 12.5 | 156 | 450 | 7.7 | 27.0 |
| 3 | Anti-MMP-9 | MMP-9 | 300.227 | 12.5 | 133 | 139 | 12.9 | 35.1 |
| 4 | Anti-VEGF | MMP-9 | 293.857 | 12.0 | 191 | 97 | 8.7 | 0.07 |

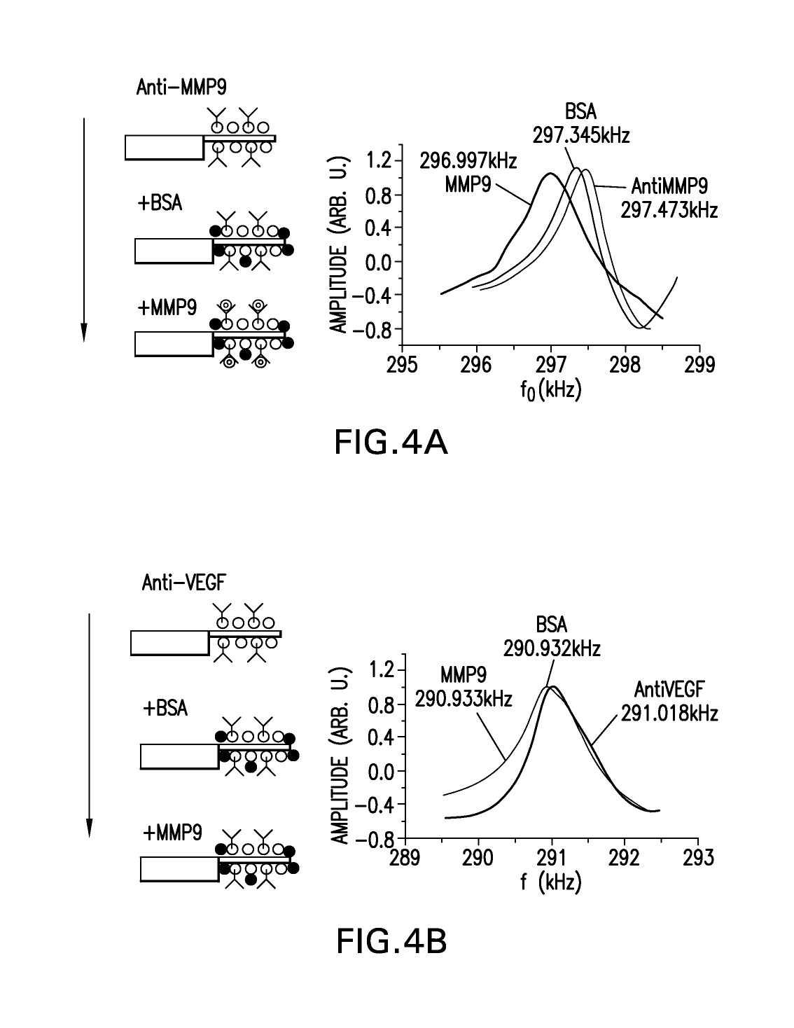

In order to further demonstrate the specificity of this technique, similar experiments were carried out using anti-MMP-9 instead of anti-VEGF. The results are shown in FIGS. 4(a)-(b) and summarized in Table I. Referring to FIGS. 4(a) and 4(b), a frequency shift is observed when anti-MMP 9 was exposed to MMP-9, whereas no significant frequency shift is observed when anti-VEGF is exposed to MMP-9. When anti-MMP-9 was used and then exposed to the MMP-9 solution, a shift in resonance frequency was observed. In this case, the changes in frequency correspond to 139 pg of anti-MMP-9 and 35 pg of MMP-9, thus resulting in antibody/antigen coverage of 76%. When another anti-VEGF-coated cantilever was exposed to MMP-9, however, the shift in the resonance frequency was extremely small, approximately 1 Hz, and well within the error of the measurement, 0.004 kHz, and in terms of mass, corresponding to 0.2 pg.

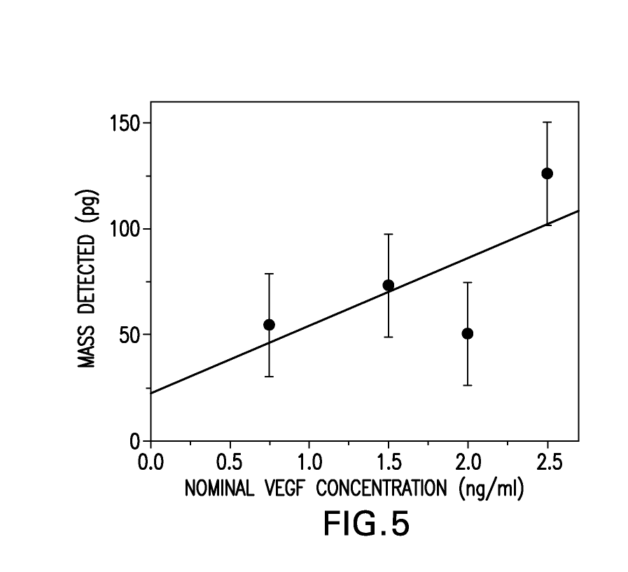

Finally, the response of the cantilevers to calibrated VEGF solutions is shown in FIG. 5. The line is a linear fit to the data with a slope of 31.8 pg/(nm/ml) and an intercept of 22.5 pg. The non-zero y-intercept is a result of VEGF binding to the support chip instead of the cantilever itself. The x-axis is the nominal concentration of the solution while the y-axis is the measured mass attached to the cantilever determined from the frequency measurements. The red line is a linear fit to the data. Error bars were calculated as the average standard deviation of all seven measurements divided by the square root of the number of measurements. The resulting sensitivity was 32 pg/(nm/ml). According to the data illustrated in FIG. 5, the sensitivity of the technique, as determined from the slope of the red line, is approximately 32 pg/(ng/ml). Given that the ultimate mass sensitivity is 0.2 pg, we estimate that the ultimate VEGF sensitivity of this technique is 0.2/32 ng/ml=6.3 pg/ml, which is similar to the ELISA sensitivity.

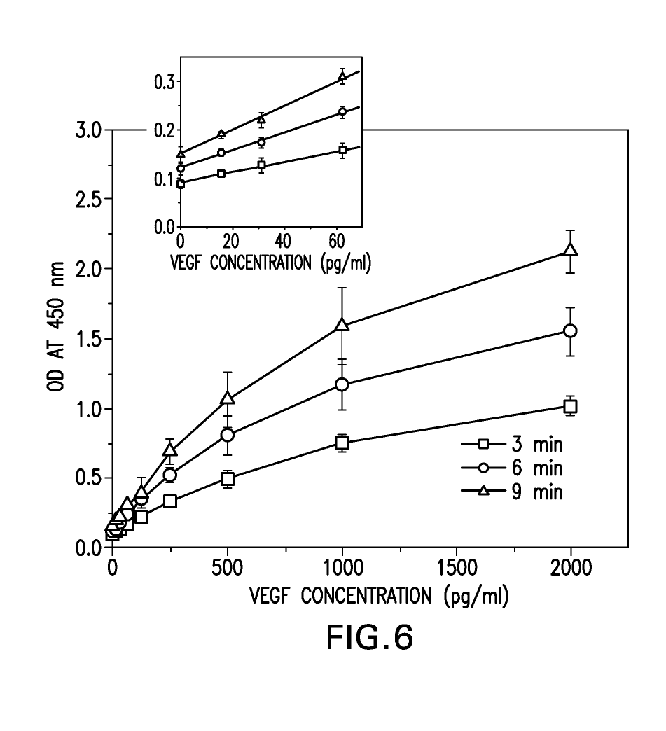

The results of the ELISA analysis showed that the optimal capture antibody dilution was 1:500, and that the optimal detection antibody dilution was 1:2000. These two dilutions gave the least amount of relative background and produced the most accurate standard curve. The results of the optimized analysis are shown in FIG. 6, from which 2,000 pg/ml was the highest point of the standard curve that could be detected by the plate reader. From the initial ELISA data it is known that the minimal detection limit could be much less than 156 pg/ml. A measure of the sensitivity comes from the fit to the straight lines shown in the inset of FIG. 6 for small VEGF concentrations where the ELISA response is linear. Error bars correspond to the standard deviation of six separate measurements. The lines are guides to the eye. In the inset of FIG. 6, the lines are linear fits to the data in the range shown. Using standard error propagation techniques, the uncertainty in the determination of the VEGF concentration C given by a linear optical response R=a+bC is given by Equation (3):

$${σ}_{C} = \frac{\sqrt{{σ}_{a}^{2} + {σ}_{R}^{2} + {( ( R - a ) {σ}_{b} / b )}^{2}}}{b} \qquad ( 3 )$$

where a and b are the intercept and slope, respectively, of the fits shown in the inset of FIG. 6, whereas σₐ and σ_{b} are the uncertainties of these parameters resulting from the fits. The value for the optical response R used for the calculation was the average in the linear range. The results are summarized in Table II. Referring to Table II, which contains the results for ELISA calibration experiments where the response is linear, the minimum detectible concentration was 7 pg/ml. (see FIG. 6). The linear fits, shown in the inset of FIG. 6, were performed taking into account the uncertainty of each data point, from which the uncertainty of the fitting parameters (slope and y-intercept) were obtained. The sensitivity was obtained using standard error propagation techniques resulting in Equation 3.

TABLE II
A summary of the results for ELISA calibration experiments
where the response is linear.

| Incubation | Y-Intercept | | Sensitivity |
|---|---|---|---|
| Time (min) | (OD) a | Slope (10⁻³ OD/pg/ml) b | (pg/ml) |
| 3 | 0.0913 ± .0022 | 1.05 ± 0.05 | 14.5 |
| 6 | 0.1227 ± 0.0036 | 1.80 ± 0.13 | 8.8 |
| 9 | 0.1515 ± 0.0038 | 2.46 ± 0.17 | 6.7 |

It has been shown that it is possible to link anti-VEGF and anti-MMP-9 antibodies to a silicon cantilever surface. The measurement in air of the shift of cantilever resonance frequency due to specific VEGF binding with anti-VEGF allows the detection of VEGF in a solution. This provides a significant advantage because measurements in air yield a much higher resonance frequency and a sharper resonance profile, both of which improve the sensitivity of device by at least one order of magnitude.

The specificity of the binding interaction was demonstrated by exposing anti-VEGF-coated microcantilevers to ferritin and MMP-9, where no statistically significant frequency shifts were detected. On the other hand, an anti-MMP-9-coated cantilever was sensitive to the presence of MMP-9, demonstrating the universality of this technique as applied to early cancer detection. This means that no cross-talk between anti-VEGF and MMP-9 binding was measured and perhaps more importantly, also demonstrates that the binding at the cantilever is specific. In another embodiment, MMP-9 can be detected via microcantilevers using similar methodology as that used for VEGF detection. With this technique it has been determined that the mass sensitivity of the technique can be about 0.2 pg. The main problem with this technique is that in order to detect small concentrations and small volumes, specific binding must only occur on the cantilever tip and not on the entire cantilever beam and supporting chip or base.

### Improved Sensitivity

In order to achieve ultimate sensitivity, that is, in sample solutions with low concentrations and small volumes, which is greater than other detection methods that place the receptor compound over the entire cantilever base and beam, the receptor compound, e.g. anti-VEGF, should only be placed at the distal-most end of the cantilever beam, i.e., the tip of the cantilever beam. One way to achieve increased sensitivity is to coat only a portion of the cantilever beam, for example, only the top surface of the distal-most end of the cantilever beam, with a metallic coating, such as a bilayer of chromium and gold, and then use thiol-based chemistry to selectively bind a bifunctional receptor compound with specific binding affinity to the analyte only to the gold coating.

The methodology of selecting a bifunctional receptor compound that selectively adheres to only the metallic coating disposed on a first surface of a distal-most end of a cantilever beam and provides specific binding to the targeted analyte can provide significant advantages over other methods, e.g., improving the sensitivity and selectivity of the microcantilever sensor, simplifying the functionalization and chemical binding process, and minimizing effective changes in stiffness of the cantilever. The sensitivity is improved because the receptor compound is immobilized only to the metallic coating on the top surface of the distal-most end of the cantilever beam, which is the most sensitive portion of the microcantilever sensor. In addition, any measured change in the height or thickness of the microcantilever can be attributed to the analyte because the receptor compound is only bound to a small portion of the microcantilever, i.e., the distal-most end of the cantilever beam, and the overall height or thickness of the entire cantilever is not significantly changed. The functionalization and chemical binding process is simplified because immobilizing a bifunctional receptor compound directly to the cantilever surface via the metallic coating eliminates the need for an additional compound to bind to the analyte, e.g., APTES, glutaraldehyde, or PA. Another benefit of immobilizing the receptor compound to only the distal-most end of the cantilever beam via the metallic coating is that effective changes in the stiffness of the cantilever are minimized. In contrast, the binding of the metallic coating, sensing elements, and analytes to the entire cantilever would increase effective changes in the stiffness of the cantilever.

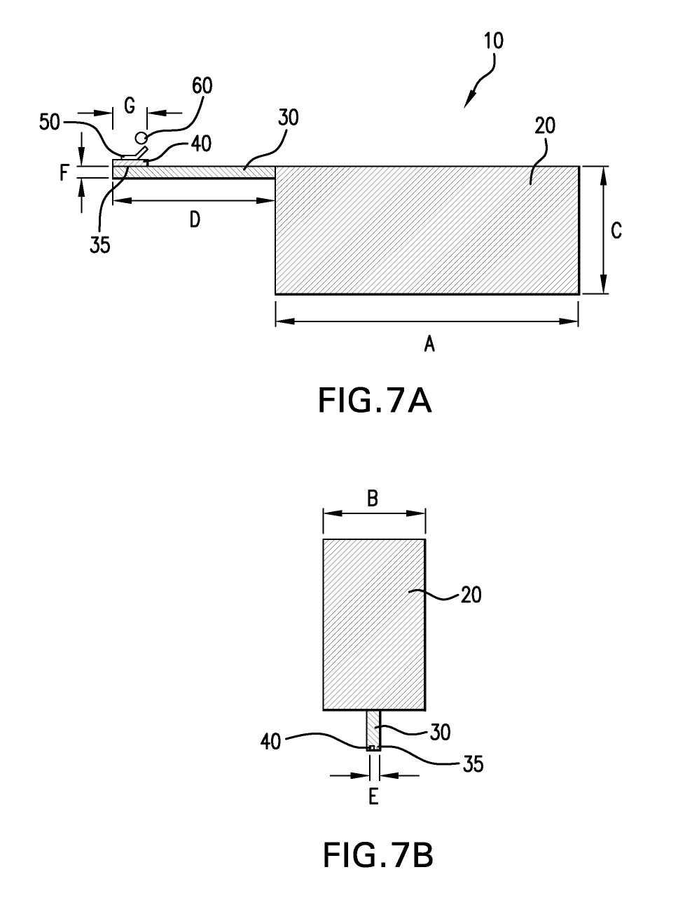

Referring to FIGS. 7(a) and 7(b), embodiments of a microcantilever sensor for targeted analyte detection can generally comprise a microcantilever 10 having a base 20 and a beam 30, a metallic coating 40 disposed substantially only on a first surface 35 of a distal-most end of the beam 30, a receptor compound 50 immobilized to the metallic coating 40 wherein the receptor compound 50 has substantially exclusive binding interaction with the analyte 60. The receptor compound 50 can be a bifunctional compound having a linking site that can bind the receptor compound 50 to the metallic coating 40 and an active receptor site with specific binding affinity for the analyte 60, for example, a thiol-terminated biochemical receptor compound. The bifunctional receptor compound can be an antibody, an isolated Fab′ fragment, a DNA fragment, a RNA fragment, an aptamer, a protein, or a carbohydrate. The metallic coating 40 can be a noble metal or a semi-noble metal, for example, chromium, gold, copper, platinum, silver, iridium, ruthenium, palladium, and combinations thereof. In certain embodiments, the metallic coating 40 can be a bilayer of chromium and gold.

Referring to FIGS. 7(a) and 7(b), in embodiments of the functionalized microcantilever, the length (A), width (B), and height (C) of the cantilever base 20 can be 2.5 mm×2 mm×0.5 mm, respectively. Similarly, in embodiments of the functionalized microcantilever, the length (D), width (E), and height (F) of the cantilever beam 30 can be 90 μm×35 μm×2 μm, respectively. In order to achieve ultimate sensitivity, the receptor compound should only be immobilized on the first surface 35 of the distal-most end of the cantilever beam 30. In certain embodiments, the metallic coating 40 can be disposed in a geometric pattern having a total area from about 100 μm² to about 144 μm², such as, for example, a square or rectangular pattern having an edge length (G) in the range of about 10 μm to about 12 μm. Increasing the edge length (G) greater than 10% of the beam length (D) will not increase the sensitivity significantly, but decreasing the edge length (G) substantially may decrease the overall sensitivity. The metallic coating 40 can be disposed on the first surface 35 of the distal-most end of the beam 30 by electron-beam lithography. The analyte 60 can be any compound having exclusive affinity for the receptor compound 50, for example, biological compounds, such as, VEGF, anti-VEGF, MMP-9, anti-MMP-9, and ferritin. In further embodiments, the microcantilever sensor can comprise a plurality of microcantilevers disposed in an array (not shown).

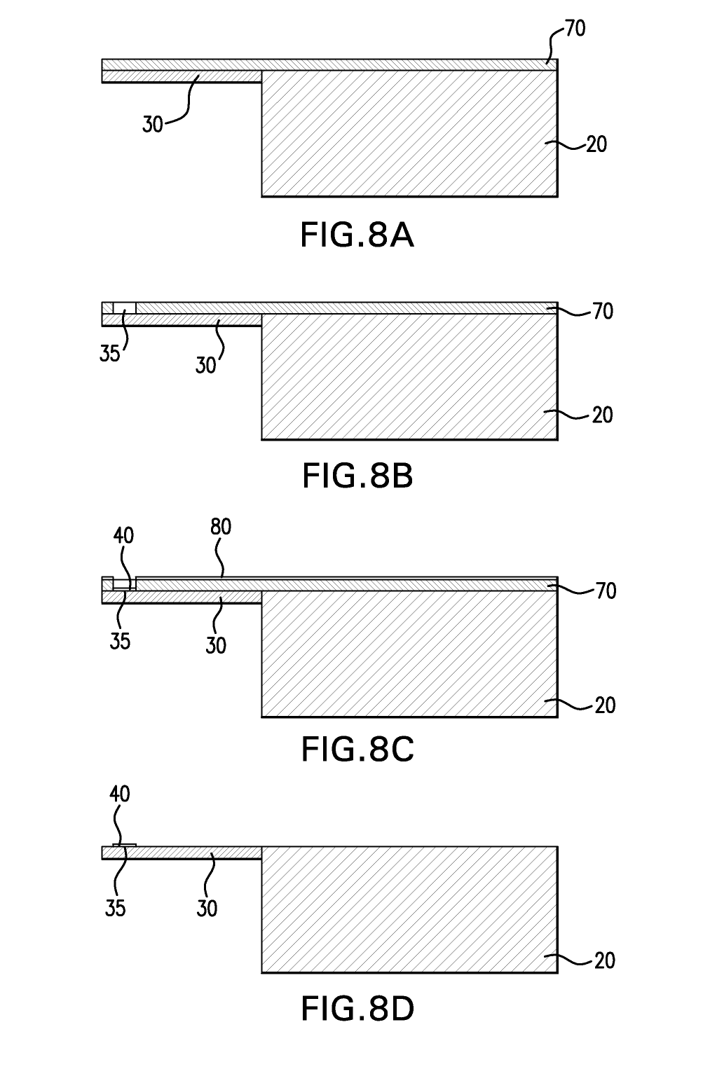

Referring to FIGS. 8(a)-(d), electron-beam lithography can be used to dispose a metallic coating 40 on the top surface 35 of the distal-most end of a cantilever beam 30. More specifically, referring to FIG. 8(a), a poly(methyl methacrylate) (PMMA) film 70 with a thickness of approximately 200 nm can be disposed onto a clean substrate by spin coating 4% PMMA in anisole at 7500 rpm for 30 seconds and baking at 150° C. for about 3 minutes. A geometric pattern, e.g., a square or rectangular pattern, can be generated on the surface by using an electron microscope to expose PMMA under the following conditions: (a) energy: 30 keV; (b) magnification: 1000×; and (c) beam current: 30 pA. In certain embodiments, a large square with an edge length of approximately 12 μm can be generated under the following conditions: (a) dwell time: 46.485 μs and (b) area dose: 55 μC/cm². In other embodiments, a small square with an edge length of approximately 10 μm can be generated under the following conditions: (a) dwell time: 101.42 μs and (b) area dose: 120 μC/cm². A significant distortion of the shape of the pattern may result if the electron microscope is out of focus while writing the pattern. Since the PMMA film can be uneven at the end, the electron microscope should be focused on one of the longer edges of the cantilever even though this means being out of focus on the rest of the cantilever.

Referring to FIG. 8(b), the PMMA film 70 can be developed under the following conditions: (a) soaking 30 seconds in 1:3 methyl isobutyl ketone (MIBK): isopropyl alcohol (IPA); (b) soaking 30 seconds in IPA; and (c) drying with nitrogen gas. The PMMA film 70 should be viewed under a microscope to determine whether some additional development is needed depending on how long the PMMA was baked and how much time passed between spin coating and writing.

Referring to FIG. 8(c), a metallic coating can be deposited over the PMMA mask 80 and the exposed top surface 35 of the distal-most end of the beam 30. In certain embodiments, the metallic coating can comprise a bilayer of chromium and gold in which an approximately 3 nm thick chromium film can be deposited at 0.20 A/s over both the PMMA mask 70 and exposed top surface 35 of the distal-most end of the beam 30 via thermal evaporation process, which can be followed by depositing an approximately 15 nm thick gold film at 0.5-0.6 A/s over the chromium film. The chromium coating is necessary to improve the adhesion of the gold coating to the silicon surface.

Referring to FIG. 8(d), then the PMMA mask 70 can be removed by soaking for approximately 5 minutes in acetone in a lift-off process, followed by an approximately 30 seconds rinse in methanol or IPA, thereby resulting in a gold metallic coating 40 disposed on the top silicon surface 35 of the distal-most end of the microcantilever 10.

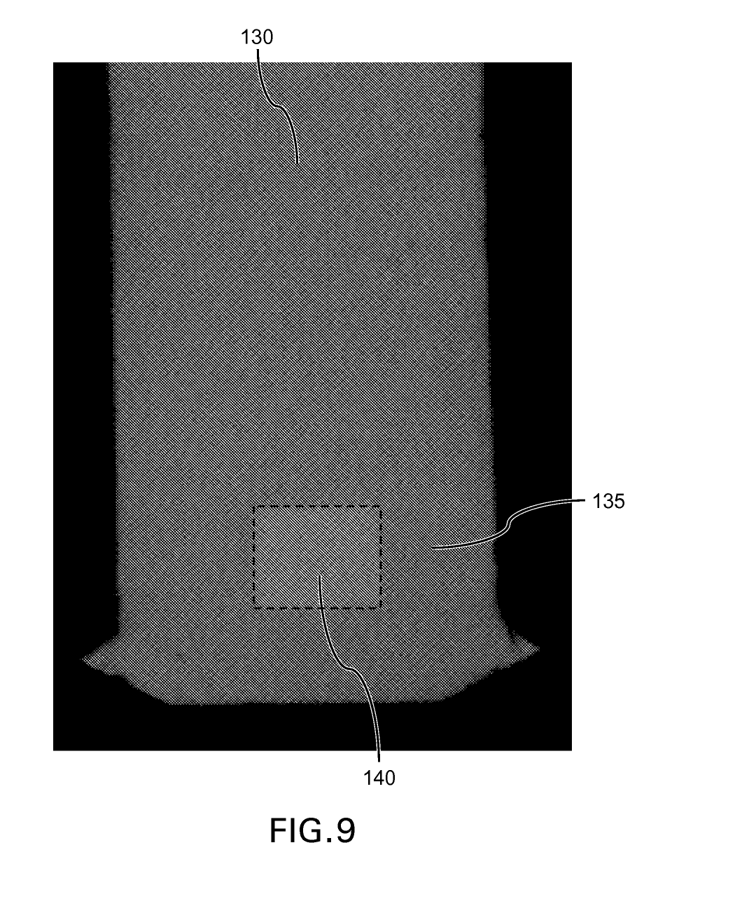

The sensitivity of the microcantilever sensor can be increased by defining the metallic coating 40 as far away as possible from the supporting edge of the cantilever base 20. Referring to FIG. 9, as described above, in certain embodiments, electron-beam lithography can be used to define and deposit a gold pattern 140 only on the first surface 135 of the distal-most end of the microcantilever beam 130, such as, for example, a 10 μm×10 μm gold pattern. The sensitivity is improved because the receptor compound is immobilized only to the metallic coating on the top surface of the distal-most end of the cantilever beam, which is the most sensitive portion of the microcantilever sensor, and not the entire cantilever.

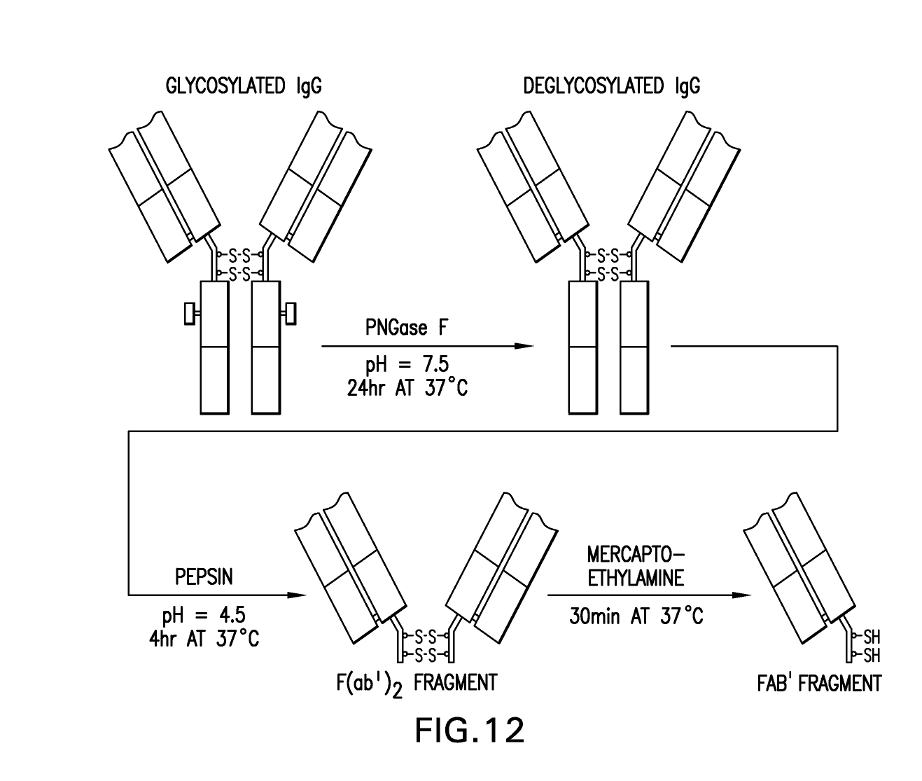

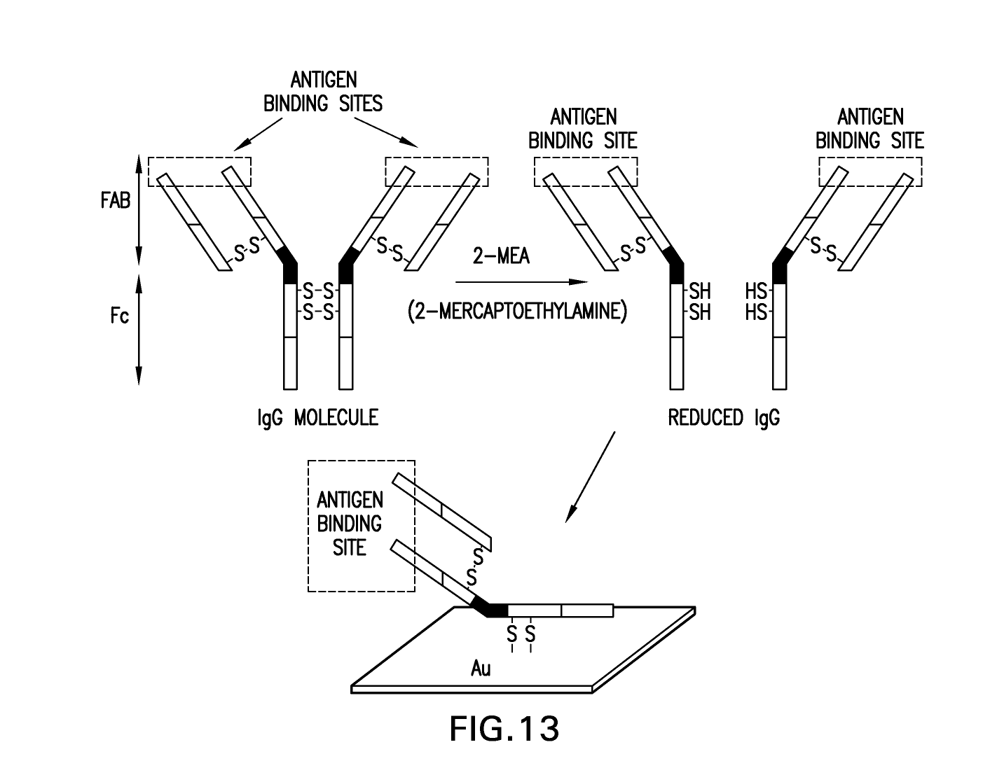

In order to ensure that the receptor compound binds only to the metallic coating disposed on the first surface of the distal-most end of the cantilever beam, the receptor compound can be a bifunctional compound. The bifunctional compound can have a terminal receptor site with specific binding affinity for the analyte and a terminal linking site that tends to preferentially bind to the metallic coating instead of the silicon surface of the cantilever. In certain embodiments, the linking site can be, for example, a thiol functional group (S—S or S—H) or a functional group that can bind to a linking compound having a thiol group immobilized on the metallic coating, e.g., HS—R—NH₂ and HS—R—COOH wherein R can be alkyl or aryl in the case of gold or platinum and an isocyanate in the case of platinum. See, e.g., Martin, B. R., Dermody, D. J., Reiss, B. D., Fang, M., Lyon, L. A., Natan, M. J., Mallouk, T. E. “Orthogonal Self-Assembly on Colloidal Gold-Platinum Nanorods” Advanced Materials, 11:1021-1025 (1999). In certain embodiments, the receptor compound can be, for example, an antibody that has been subjected to the enzymatic processes of deglycosylation and pepsinolysis to isolate Fab′ fragments of the antibody. Recall that Fab′ fragments have a terminal epitope where the antigen binds to the antibody and a terminal thiol group that can bind to a gold surface. A process of generating a Fab′ fragment from an antibody and immobilizing the terminal thiol group of the Fab′ fragment to a gold surface is illustrated in FIGS. 12 and 13. See, e.g., A. A. Karyakin, G. V. Presnova, M. Y. Rubtsova, and A. M. Egorov, “Oriented Immobilization of Antibodies into the Gold Surfaces via Their Native Thiol Groups”, Anal. Chem. 72, 3805-3811 (2000), which is hereby incorporated by reference in its entirety.

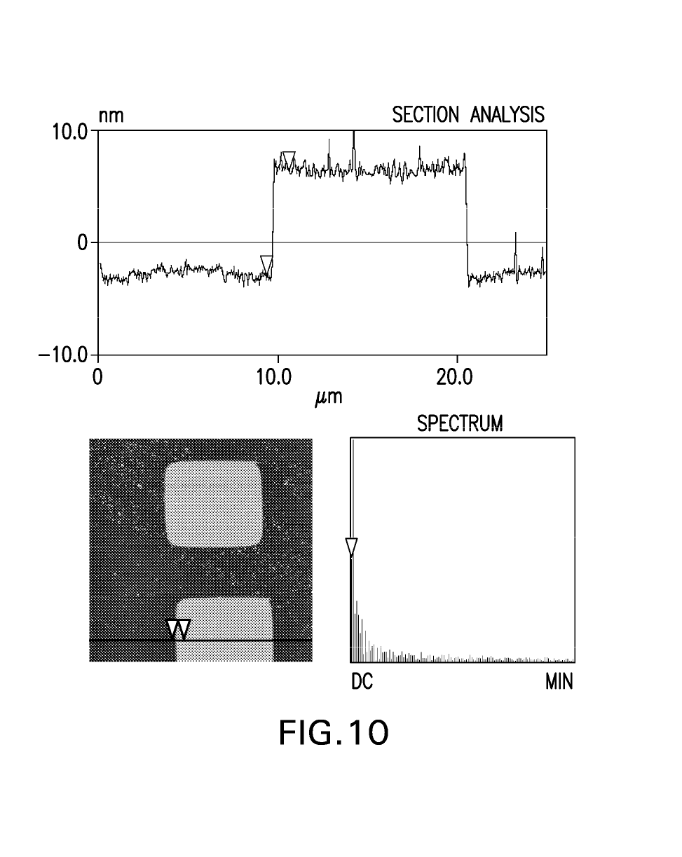

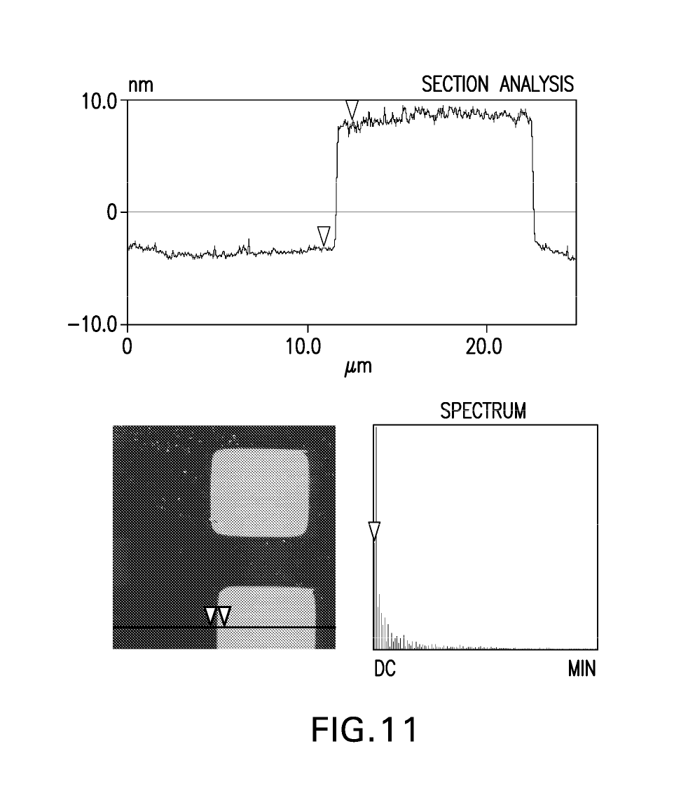

The enzymatic processes of deglycosylation and pepsinolysis used to isolate Fab′ fragments and the process of immobilizing Fab′ fragments to a gold coated silicon surface was verified by immobilizing an organic molecule with a terminal thiol functional group, e.g., DTSP, on a gold pattern that was deposited on a bare silicon wafer. As shown in FIGS. 10 and 11, the measured increase in height is equivalent to the height of a DTSP monolayer. An atomic force microscopy image of a 10 μm×10 μm×9.3 nm gold pad deposited on a silicon wafer is shown in FIG. 10. An atomic force microscopy image of the gold pad deposited on the silicon wafer of FIG. 10 after depositing DTSP on the gold surface is shown in FIG. 11. FIG. 11 shows that the height of the square increased to 11.5 nm after depositing DTSP onto the gold surface, or the height equivalent to a DTSP monolayer.

In certain embodiments, a bifunctional receptor compound with a terminal thiol group as a linking site and an active receptor site with specific binding affinity for the analyte can be created from an antibody that has been subjected to the enzymatic processes of deglycosylation and pepsinolysis to isolate Fab′ fragments of the antibody. For example, whole IgG can be cleaved near the hinge region via pepsinolysis to release an active Fab′ fragment. The terminal thiol group of the Fab′ fragment can be a linking site. Prior to cleavage, the IgG may remain glycosylated, or may be deglycosylated via peptide N-glycosidase F (PNGaseF, available from NEW ENGLAND BIOLABS, INC.). The released Fab′ fragments are separated from Fc fragments and remaining whole IgG using immobilized protein A or immobilized protein G. The remaining Fab′ fragment is reduced using mercaptoethylamine (2-MEA) and then separated from the reducing agent using a molecular weight cutoff filter. The Fab′ fragments are then incubated with the gold surface and immobilized only to the gold surface. The terminal thiol group of the Fab′ fragment that resulted from treatment of Fab′ with 2-MEA can bind to the gold surface.

A high purity solution of Fab′ fragments can be prepared from the IgG1 form of the antibody anti-VEGF. See, e.g., Wilson, D. S., Wu, J., Peluso, P., Nock, S. “Improved method for pepsinolysis of mouse IgG1 molecules to F(ab′)₂ fragments” Journal of Immunological Methods, 260 (2002) 29-36, which is hereby incorporated by reference in its entirety. These fragments were specially prepared for use on gold surfaces, which consisted of four separate procedures intended to be completed in sequence: (a) pepsinolysis of whole IgG; (b) removal of whole IgG and Fc fragments with protein A or protein G; (c) reduction of F(ab′)₂ to Fab′ with 2-MEA; and (d) final purification, removal of 2-MEA, and concentration. The entire procedure can be completed in a minimum of 1.5-2 days. Please note that only glass should be used in this process because plastic may cause proteins to stick.

The purpose of pepsinolysis of whole IgG is to cleave the IgG1 molecule below the disulfide bonds holding the two heavy chains together, resulting in a F(ab′)₂ fragment. Immobilized pepsin can be used for its increased activity, higher purity, and easy removal from solution. Pepsinolysis requires a cleavage buffer, e.g., 20 mM sodium acetate, 20 mM TRIS buffer to pH 4, and immobilized pepsin (available from PIERCE CHEMICALS, product number 20343). Whole IgG can be cleaved by placing 50 μL of immobilized pepsin into a glass or borosilicate vial. Vials containing a bulbous end are especially helpful because they tend to trap the beads when centrifuged, which makes the removal of samples much easier. The storage liquid can be removed from the beads and washed several times with pH 4 cleavage buffer by spinning down, removing the liquid, and resuspending with fresh buffer. After the final wash, the beads should not be resuspended. To the deglycosylated IgG1, 50 μL of cleavage buffer can be added and the pH can be adjusted to pH 4.0, if necessary. Because pepsin is irreversibly denatured at pH levels at or above 6, the total pH must be kept low. This solution can be combined with the bead solution, mixed well by pipetting, and incubated with the vial on its side for 16-20 hours at 37° C. to prevent the immobilized pepsin from migrating to the bottom and isolating itself from the IgG solution. The recommended samples taken for SDS-PAGE analysis can include a 4.2 μL sample taken at 6-10 hours of incubation (0.2 μg total) and a 4.2 μL sample taken at end of incubation (0.2 μg total).

The removal of unreacted IgG1 and large Fc fragments can be accomplished by incubating the pepsinolysis sample with protein A or protein G. This genetically engineered protein improves binding efficiency with murine hosted antibodies, but still requires that a great excess of the bead be used for efficient purification. This process requires immobilized protein A or protein G, (available from PIERCE CHEMICALS, product number 20421), IMMUNOPURE protein A binding buffer, and IgG elution buffer (available from PIERCE CHEMICALS, product number 21004) or 0.1 M glycine at pH 2-3. Purification can be accomplished by placing 100 μL of immobilized protein A or protein G into the glass or borosilicate vial. This can be spun down and the storage solution can be taken off. This can be washed several times with the binding buffer in the same manner as the immobilized pepsin. The beads should not be resuspended after the final wash. The IgG solution can be buffered to a pH of about 7. The IgG can be combined with the beads, mixed well, and incubated on the vial's side for 90-120 minutes. This can be spun down. The liquid should contain only F(ab′)₂ and any Fc fragments unable to bind. To remove what has been captured by protein A or protein G, 100 μL of elution buffer or 0.1 M glycine can be added. This can be incubated for 5 minutes at room temperature, spun down, and the solution can be collected. This should contain unreacted IgG and Fc fragments able to bind to the protein A or protein G. The recommended samples taken for SDS-PAGE analysis that can be seen on gel will depend on extent of deglycosylation and pepsinolysis, but can include a 10 μL sample of “flow through” taken from incubation step (0.6 μg total-ideally) and a 10 μL sample of elution.

The reduction of F(ab′)₂ to Fab′ can be accomplished with 2-mercaptoethylamine (2-MEA). 2-MEA may be used to selectively reduce the disulfide bonds holding the two Fab′ fragments together forming F(ab′)₂ without causing a great risk to the disulfide bonds holding together the light and heavy chains. For analysis by various methods, an alkylation step has been documented in this protocol, but should not be used on the final product that will be immobilized. The required products for this process include 2-MEA, also commonly known as cysteamine, and iodoacetic acid, if alkylation is being performed. The disulfide bonds can be reduced by creating a solution of 1 M 2-MEA. This solution should be made fresh for every preparation. A 10 mg/ml solution should receive a final concentration of 50 mM. At this step, the total protein concentration can be less than 0.044 mg/ml, so enough of the 2-MEA solution should be added to achieve a final concentration of 0.42 mM. This needs to be sealed well and incubated for 90 minutes at 37° C. Alkylation can be performed on samples taken from the reduction by adding sufficient iodoacetic acid to give a final concentration of 50 mM. This concentration should be sufficient to alkylate the newly formed Fab′ fragments as well as the 2-MEA in solution stopping the reaction. The recommended samples taken for SDS-PAGE analysis can include a 10 μL of sample taken after 90 minutes incubation.

The final purification of Fab′ samples removes 2-MEA, salts, and the small Fc fragments generated by the pepsinolysis described above. The filter material used can be regenerated cellulose which should not result in the produced thiols' sticking to it. The remaining product only consists of Fab′ that should be suitable for incubation on gold. The required products for this process include MILLIPORE MICROCON Centrifugal Filter Devices (10 kDa MWCO). Purification is accomplished by flushing the device several times with deionized water by adding 500 μL to the reservoir and centrifuging until entire volume has passed through filter. Freshly reduced Fab′ can be added and centrifuged until the bulk of the solution has passed through and the volume of the reservoir remains constant. If the reservoir is centrifuged until dryness, a recorded amount of deionized water should be added, agitated for 30 seconds, and reserve the liquid. The recommended samples taken for SDS-PAGE analysis can include a 10 μL sample of purified sample.

The terminal thiol group of the Fab′ fragments can be immobilized to a gold surface. The gold surface should be cleaned before immoblizing the Fab′ fragments. The gold coated cantilever can be immersed in a piranha etch (a 3:1 H₂SO₄:H₂O₂ solution) for ten minutes at room temperature to dissolve most organic contaminants from the gold surface. Then the gold surface can be washed with distilled filtered water. Then the cantilever can be immersed in the purified Fab′ sample and incubated at room temperature for one hour.

Referring again to FIGS. 7(a) and 7(b), embodiments of a method for functionalizing a microcantilever sensor for targeted analyte detection can generally comprise disposing a metallic coating substantially only on a first surface 35 of a distal-most portion of a microcantilever 10 and and then immobilizing a receptor compound 50 to the metallic coating 40 wherein the receptor compound 50 can have substantially exclusive binding interaction with the analyte 60. The receptor compound 50 can be a biochemical receptor compound, for example, a thiol-terminated bifunctional compound having an active receptor site with specific binding affinity for the analyte. The bifunctional receptor compound can be an antibody, an isolated Fab′ fragment, a DNA fragment, a RNA fragment, an aptamer, a protein, a carbohydrate, or DTSP. The metallic coating 40 can be at least one of a noble metal and a semi-noble metal, for example, chromium, gold, copper, platinum, silver, iridium, ruthenium, palladium, and combinations thereof. In certain embodiments, the metallic coating 40 can be a bilayer of chromium and gold. In order to achieve ultimate sensitivity, in certain embodiments the metallic coating 40 can be disposed in a pattern having a total area from about 100 μm² to about 144 μm², such as, for example, a square or rectangular pattern having an edge length in the range of about 10 μm to about 12 μm. The metallic coating 40 can be disposed on the first surface 35 of the distal-most end of the beam 30 by electron-beam lithography. The analyte 60 can be any compound having exclusive affinity for the receptor compound 50, for example, biological compounds, such as VEGF, anti-VEGF, MMP-9, anti-MMP-9, and ferritin.

Embodiments of a method for using a microcantilever sensor for detecting a targeted analyte can generally comprise treating a first surface 35 of a distal-most end the microcantilever 10 with a metallic compound 40, immobilizing a receptor compound 50 to the metallic compound 40 wherein the receptor compound 50 can have substantially exclusive binding interaction with the targeted analyte 60, exposing the first surface 35 to a sample solution containing the analyte 60, drying the first surface 35, and resonating the microcantilever 10 in air or vacuum to detect the analyte 60. The receptor compound 50 can be a biochemical receptor compound, for example, a thiol-terminated bifunctional compound having an active receptor site with specific binding affinity for the analyte. The bifunctional receptor compound can be an antibody, an isolated Fab′ fragment, a DNA fragment, a RNA fragment, an aptamer, a protein, a carbohydrate, or DTSP. The metallic coating 40 can be at least one of a noble metal and a semi-noble metal, for example, chromium, gold, copper, platinum, silver, iridium, ruthenium, palladium, and combinations thereof. In certain embodiments, the metallic coating 40 can be a bilayer of chromium and gold. In order to achieve ultimate sensitivity, in certain embodiments the metallic coating 40 can be disposed in a pattern having a total area from about 100 μm² to about 144 μm², such as, for example, a square or rectangular pattern having an edge length in the range of about 10 μm to about 12 μm. The metallic coating 40 can be disposed on the first surface 35 of the distal-most end of the beam 30 by electron-beam lithography. The analyte 60 can be any compound having exclusive affinity for the receptor compound 50, for example, biological compounds, such as VEGF, anti-VEGF, MMP-9, anti-MMP-9, and ferritin.

The microcantilever sensor described above could be improved by, for example, measuring in a controlled vacuum environment instead of air. Measuring in a controlled vacuum environment would increase the Q-factor, or sharpness of the resonance, thus increasing the ultimate sensitivity of the measurement, and improve the reproducibility between measurements by providing nominally identical environmental conditions for each measurement. It is also known that in nominal vacuum conditions, biomolecular compounds retain water molecules that keep them metabolically active. A vacuum environment of an air pressure of approximately 1 Torr could be sufficient for this purpose. At these pressures, the biomolecules may retain their structure since they will not be totally dehydrated.

Therefore, what has been described above includes exemplary embodiments of a functionalized microcantilever sensors and associated methods for targeted analyte detection. It is, of course, not possible to describe every conceivable combination of components or methodologies for purposes of this description, but one of ordinary skill in the art may recognize that further combinations and permutations are possible in light of the overall teaching of this disclosure. Accordingly, the description provided herein is intended to be illustrative only, and should be considered to embrace any and all alterations, modifications, and/or variations that fall within the spirit and scope of the appended claims.

What is claimed is:

1. A microcantilever sensor for targeted analyte detection comprising:
a. a microcantilever having a base and a beam;
b. a metallic coating disposed substantially only on a first surface of a distal-most end of said beam;
c. a receptor compound immobilized to said metallic coating; and
d. said receptor compound having substantially exclusive binding interaction with said analyte.

2. The microcantilever sensor of claim 1, wherein said receptor compound is a thiol-terminated bifunctional compound having an active receptor site with specific binding affinity for said analyte.

3. The microcantilever sensor of claim 2, wherein said receptor compound is a biochemical receptor compound.

4. The microcantilever sensor of claim 3, wherein said biochemical receptor compound is selected from the group consisting of an antibody, an isolated Fab′ fragment, a DNA fragment, a RNA fragment, an aptamer, a protein, a carbohydrate, and DTSP.

5. The microcantilever sensor of claim 1, wherein said metallic coating is at least one of a noble metal and a semi-noble metal.

6. The microcantilever sensor of claim 1, wherein said metallic coating is selected from the group consisting of chromium, gold, copper, platinum, silver, iridium, ruthenium, palladium, and combinations thereof.

7. The microcantilever sensor of claim 1, wherein said metallic coating comprises a bilayer of chromium and gold.

8. The microcantilever sensor of claim 1, wherein said metallic coating is disposed in a pattern having a total area from about 100 μm² to about 144 μm².

9. The microcantilever sensor of claim 1, wherein said metallic coating is disposed in a square or rectangular pattern having an edge length in the range of about 10 μm to about 12 μm.

10. The microcantilever sensor of claim 1, wherein said metallic coating is applied to said microcantilever by electron-beam lithography.

11. The microcantilever sensor of claim 1, further comprising a plurality of said microcantilevers disposed in an array.

12. The microcantilever sensor of claim 1, wherein said analyte is a protein selected from the group consisting of VEGF, anti-VEGF, MMP-9, anti-MMP-9, and ferritin.

13. A method for functionalizing a microcantilever sensor for targeted analyte detection, the method comprising:
a. disposing a metallic coating substantially only on a first surface of a distal-most end of said microcantilever; and
b. immobilizing a receptor compound to said metallic coating, said receptor compound having substantially exclusive binding interaction with said analyte.

14. The microcantilever sensor of claim 13, wherein said receptor compound is a thiol-terminated bifunctional compound having an active receptor site with specific binding affinity for said analyte.

15. The microcantilever sensor of claim 14, wherein said receptor compound is a biochemical receptor compound.

16. The microcantilever sensor of claim 15, wherein said biochemical receptor compound is selected from the group consisting of an antibody, an isolated Fab′ fragment, a DNA fragment, a RNA fragment, an aptamer, a protein, a carbohydrate, and DTSP.

17. The microcantilever sensor of claim 13, wherein said metallic coating is at least one of a noble metal and a semi-noble metal.

18. The microcantilever sensor of claim 13, wherein said metallic coating is selected from the group consisting of chromium, gold, copper, platinum, silver, iridium, ruthenium, palladium, and combinations thereof.

19. The microcantilever sensor of claim 13, wherein said metallic coating comprises a bilayer of chromium and gold.

20. The microcantilever sensor of claim 13, further comprising disposing said metallic coating in a pattern having a total area from about 100 μm² to about 144 μm².

21. The microcantilever sensor of claim 13, further comprising disposing said metallic coating in a square or rectangular pattern having an edge length in the range of about 10 μm to about 12 μm.

22. The microcantilever sensor of claim 13, further comprising applying said metallic coating to said microcantilever by electron-beam lithography.

23. The microcantilever sensor of claim 13, wherein said analyte is a protein selected from the group consisting of VEGF, anti-VEGF, MMP-9, anti-MMP-9, and ferritin.

24. A method for using a microcantilever sensor for detecting a targeted analyte, the method comprising:
a. treating a first surface of a distal-most end of said microcantilever with a metallic compound;
b. immobilizing a receptor compound to said metallic compound, said receptor compound having substantially exclusive binding interaction with said targeted analyte;
c. exposing said first surface to a sample solution containing said targeted analyte;
d. drying said first surface; and
e. resonating said microcantilever in air or vacuum to detect said targeted analyte.

25. The microcantilever sensor of claim 24, wherein said receptor compound is a thiol-terminated bifunctional compound having an active receptor site with specific binding affinity for said analyte.

26. The microcantilever sensor of claim 25, wherein said receptor compound is a biochemical receptor compound.

27. The microcantilever sensor of claim 26, wherein said biochemical receptor compound is selected from the group consisting of an antibody, an isolated Fab′ fragment, a DNA fragment, a RNA fragment, an aptamer, a protein, a carbohydrate, and DTSP.

28. The microcantilever sensor of claim 24, wherein said metallic compound is at least one of a noble metal and a semi-noble metal.

29. The microcantilever sensor of claim 24, wherein said metallic compound is selected from the group consisting of chromium, gold, copper, platinum, silver, iridium, ruthenium, palladium, and combinations thereof.

30. The microcantilever sensor of claim 24, wherein said metallic compound comprises a bilayer of chromium and gold.

31. The microcantilever sensor of claim 24, further comprising disposing said metallic compound in a pattern having a total area from about 100 μm² to about 144 μm².

32. The microcantilever sensor of claim 24, further comprising disposing said metallic compound in a square or rectangle pattern having an edge length in the range of about 10 μm to about 12 μm.

33. The microcantilever sensor of claim 24, applying said metallic compound to said microcantilever by electron-beam lithography.

34. The microcantilever sensor of claim 24, wherein said analyte is a protein selected from the group consisting of VEGF, anti-VEGF, MMP-9, anti-MMP-9, and ferritin.
